(19) United States
(12) Patent Application Publication
(10) Pub. No.: US 2007/0049889 A1
(43) Pub. Date: Mar. 1, 2007

(54) Printed absorbent articles

(76) Inventors: Todd C. Larson, Grayslake, IL (US); Matthew T. Fitton, Appleton, WI (US)

Correspondence Address: KIMBERLY-CLARK WORLDWIDE, INC., 401 NORTH LAKE STREET, NEENAH, WI, 54956, US

(21) Appl. No.: 11/217,261

(22) Filed: Aug. 31, 2005

(51) Int. Cl. A61F 13/15

(52) U.S. Cl. 604/380; 604/361; 604/391

(57) ABSTRACT

A disposable absorbent article including a topsheet, a backsheet and an absorbent core located between the topsheet and the backsheet. Further wherein the backsheet includes a bond pattern which defines a first bond region having a first bond pattern and a first bond perimeter and a second bond region having a second bond pattern and a second bond perimeter. The first bond pattern is unique from the second bond pattern. A first graphic located within the first bond perimeter, and a second graphic located within the second bond perimeter.

### BACKGROUND OF THE INVENTION

The present invention relates to improved printing. More particularly, the present invention relates to improved printing for disposable absorbent articles.

Graphics are commonly printed on articles of manufacture to increase their aesthetic appeal. One example of this is graphics or images printed on absorbent garments, such as infant and children's diapers and training pants. The outer covers of these articles are frequently imprinted with brightly-colored images in the form of designs and characters that are pleasing to the child and to the caregiver. Images may also be printed on other absorbent articles such as feminine healthcare products, adult incontinence garments and bandages.

Printing the outer cover material or other portion of an article with an image has been carried out by flexographic printing, rotogravure printing, screen printing, offset printing, or other types of contact printing techniques. Additionally, non-contact printing systems have been used to print the outer cover material; these systems include ink jet printing, wax jet printing, bubble jet printing, laser jet printing, and the like.

The quality of graphics printed with these techniques has been limited by the substrates that conventional absorbent articles are constructed of. As a result, the printed graphics on conventional absorbent articles have not been completely satisfactory.

In view of the aforementioned problems, there arises the need for an absorbent article having high quality printed graphics.

### SUMMARY

In response to the foregoing need, the present inventor undertook intensive research and development efforts that resulted in the discovery of a disposable absorbent article. One version of the present invention includes a disposable absorbent article including a topsheet, a backsheet and an absorbent core located between the topsheet and the backsheet. Further wherein the backsheet includes a bond pattern which defines a first bond region having a first bond pattern and a first bond perimeter and a second bond region having a second bond pattern and a second bond perimeter. The first bond pattern is unique from the second bond pattern. A first graphic located within the first bond perimeter, and a second graphic located within the second bond perimeter.

Another version of the present invention provides a disposable absorbent including a topsheet a backsheet defining a backsheet length and an absorbent core located between the topsheet and the backsheet. Furthe the backsheet includes a bond pattern which defines a first bond region having a first bond pattern and a first bond perimeter and a second bond region having a second bond pattern and a second bond perimeter. The first bond pattern is unique from the second bond pattern. A first graphic located within the first bond perimeter, and a second graphic located within the second bond perimeter. The bond pattern on the backsheet having a repeat length of one-fourth, one-third, one-half or one times the backsheet length. The first bond pattern is located in a first waist region having a bond pattern area of at least 1 square inch and the second bond pattern is located in a crotch region.

Still another version of the present invention includes a disposable absorbent article including a bodyfacing surface and a garment facing surface. Further the garment facing surface comprises a bond pattern which defines a first bond region having a first unique bond pattern and a first bond perimeter, a second bond region having a second unique bond pattern and a second bond perimeter, and a third bond region having a third unique bond pattern and a third bond perimeter. A first graphic located within the first bond perimeter, a second graphic located within the second bond perimeter and a third graphic located within the third bond perimeter.

Still another version of the present invention includes a disposable article including a nonwoven. Further the nonwoven includes a bond pattern which defines a first bond region having a first bond pattern and a first bond perimeter and a second bond region having a second bond pattern and a second bond perimeter. The first bond pattern being unique from the second bond pattern. A first graphic located within the first bond perimeter, and a second graphic located within the second bond perimeter.

### DRAWINGS

The foregoing and other features, aspects and advantages of the present invention will become better understood with regard to the following description, appended claims and accompanying drawings where:

FIG. 1 illustrates a plan view of a disposable absorbent article in an unfolded, flat-out, uncontracted state (i.e., with all elastic induced gathering and contraction removed), with the bodyfacing surface of the article facing the viewer and with portions of the article partially cut away to illustrate underlying features;

FIG. 2 illustrates a plan view of a disposable absorbent article in an unfolded, flat-out, uncontracted state, with the garment facing surface of the article facing the viewer and with portions of the article partially cut away to illustrate underlying features;

FIG. 3 illustrates a plan view of a representative example of a bond pattern, such as may be used on a garment facing surface of the disposable absorbent articles of the invention;

FIG. 4 illustrates a plan view of a second representative example of a bond pattern, such as may be used on a garment facing surface of the disposable absorbent articles of the invention;

FIG. 5 illustrates a plan view of a third representative example of a bond pattern, such as may be used on a garment facing surface of the disposable absorbent articles of the invention;

FIG. 6 illustrates a plan view of a fourth representative example of a bond pattern, such as may be used on a garment facing surface of the disposable absorbent articles of the invention; and

FIG. 7 illustrates a plan view of a garment facing surface of a disposable absorbent article including bond regions.

### DESCRIPTION

The present invention relates to improved printing, and improved printing for use in disposable absorbent articles. As used herein, the term “disposable” refers to articles which are intended to be discarded after a limited use and which are not intended to be laundered or otherwise restored for reuse. The disposable absorbent articles of the present invention will be described in terms of a disposable diaper which is adapted to be worn by infants about the lower torso. It is understood that the improved printing of the present invention is equally adaptable for use with other types of disposable absorbent articles such as children's training pants, adult incontinence garments and the like.

With regard to the designated surfaces of a disposable absorbent article and its components, the various upper or bodyfacing surfaces are configured to face toward the body of the wearer when the disposable absorbent article is worn by the wearer for ordinary use. The various opposing, lower or garment facing surfaces are configured to face away from the wearer's body when the disposable absorbent article is worn by the wearer.

As used herein, reference to two materials or elements being “joined” is intended to refer to the situation wherein the two materials or elements are directly joined to one another or where they are indirectly joined to one another or where they are indirectly joined to an intermediate element. Similarly, methods of joining two materials or elements include forming the elements or materials integrally, or attaching the elements together such as through the use of adhesive bonds, sonic bonds, thermal bonds, pinning, stitching, or a variety of other attachment techniques known in the art, as well as combinations thereof.

As used herein the term “nonwoven fabric or web” means a web having a structure of individual fibers or threads which are interlaid, but not in an identifiable manner as in a knitted fabric. Nonwoven fabrics or webs have been formed from many processes such as for example, meltblowing processes, spunbonding processes, and bonded carded web processes. The basis weight of nonwoven fabrics is usually expressed in ounces of material per square yard (osy) or grams per square meter (g/m² or gsm) and the fiber diameters are usually expressed in microns. (Note that to convert from osy to gsm, multiply osy by 33.91).

As used herein the terms “sheet” and “sheet material” shall be interchangeable and in the absence of a word modifier, refer to woven materials, nonwoven webs, polymeric films, polymeric scrim-like materials, and polymeric foam sheeting.

As used herein the term “spunbond” refers to small diameter fibers which are formed by extruding molten thermoplastic material as filaments from a plurality of fine, usually circular capillaries of a spinneret with the diameter of the extruded filaments being rapidly reduced as by, for example, in U.S. Pat. No. 4,340,563 to Appel et al., U.S. Pat. No. 3,692,618 to Dorschner et al., U.S. Pat. No. 3,802,817 to Matsuki et al., U.S. Pat. No. 3,338,992 and U.S. Pat. No. 3,341,394 to Kinney, and U.S. Pat. No. 3,542,615 to Dobo et al., which are each incorporated by reference in their entirety herein.

As used herein the term “meltblown” means fibers formed by extruding a molten thermoplastic material through a plurality of fine, usually circular die capillaries as molten threads or filaments into converging high velocity gas (e.g. air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed, meltblown fibers. Such a process is disclosed, in various patents and publications, including NRL Report 4364, “Manufacture of Super-Fine Organic Fibers” by B. A. Wendt, E. L. Boone and D. D. Fluharty; NRL Report 5265, “An Improved Device For The Formation of Super-Fine Thermoplastic Fibers” by K. D. Lawrence, R. T. Lukas, J. A. Young; and U.S. Pat. No. 3,849,241, issued Nov. 19, 1974, to Butin, et al.

As used herein, the term “bond” and derivatives does not exclude intervening layers between the bonded elements that are part of the bonded structure unless the text requires a different meaning.

As used herein the term “thermal point bonding” involves passing a fabric or web of fibers to be bonded between a heated calender roll and an anvil roll. The calender roll is usually, though not always, patterned in some way so that the entire fabric is not bonded across its entire surface, and the anvil roll is usually flat. As a result, various patterns for calender rolls have been developed for functional as well as aesthetic reasons. One example of a pattern has points and is the Hansen Pennings or “H&P” pattern with about a 30% bond area with about 200 bonds/square inch as taught in U.S. Pat. No. 3,855,046 to Hansen and Pennings, incorporated herein by reference in its entirety. The H&P pattern has square point or pin bonding areas wherein each pin has a side dimension of 0.038 inches (0.965 mm), a spacing of 0.070 inches (1.778 mm) between pins, and a depth of bonding of 0.023 inches (0.584 mm). The resulting pattern has a bonded area of about 29.5%. Another typical point bonding pattern is the expanded Hansen Pennings or “EHP ”bond pattern which produces a 15% bond area with a square pin having a side dimension of 0.037 inches (0.94 mm), a pin spacing of 0.097 inches (2.464 mm) and a depth of 0.039 inches (0.991 mm). Another typical point bonding pattern designated “714” has square pin bonding areas wherein each pin has a side dimension of 0.023 inches, a spacing of 0.062 inches (1.575 mm) between pins, and a depth of bonding of 0.033 inches (0.838 mm). The resulting pattern has a bonded area of about 15%. Other common patterns include a diamond pattern with repeating and slightly offset diamonds with about a 16% bond area and a wire weave pattern looking, as the name suggests, like a window screen pattern having a bond area in the range of from about 15% to about 21% and about 302 bonds per square inch. Typically, the percent bonding area varies from around 10% to around 30% of the area of the fabric laminate web. As is well known in the art, the spot bonding holds the laminate layers together as well as imparts integrity to each individual layer by bonding filaments and/or fibers within each layer.

As used herein, the term “ultrasonic bonding” means a process performed, for example, by passing the fabric between a sonic horn and anvil roll as illustrated in U.S. Pat. No. 4,374,888 to Bornslaeger, incorporated by reference herein in its entirety.

As used herein, the term “continuous filaments”, refers to strands of continuously formed polymeric filaments having a length to diameter ratio of at least about a thousand and usually much higher. Such filaments will typically be formed by extruding molten material through a die head having a certain type and arrangement of capillary holes therein.

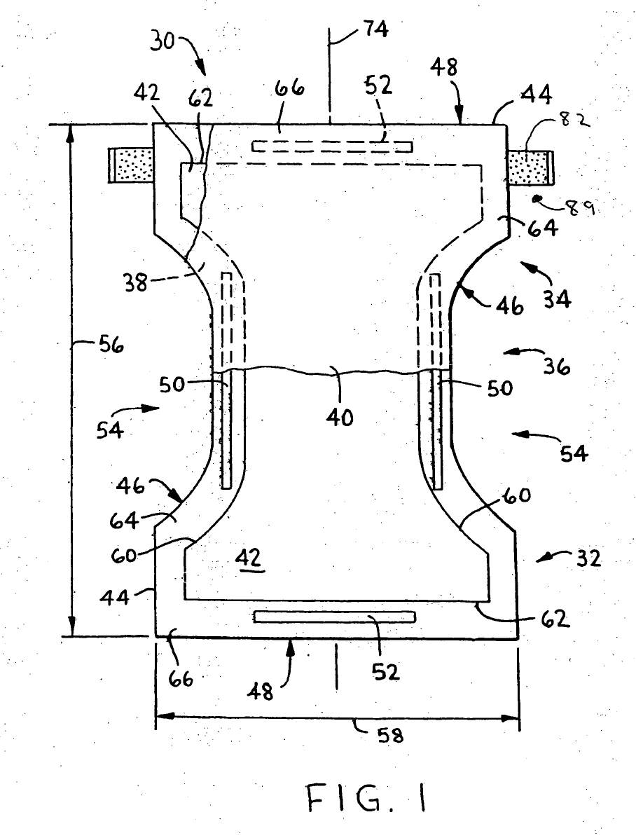

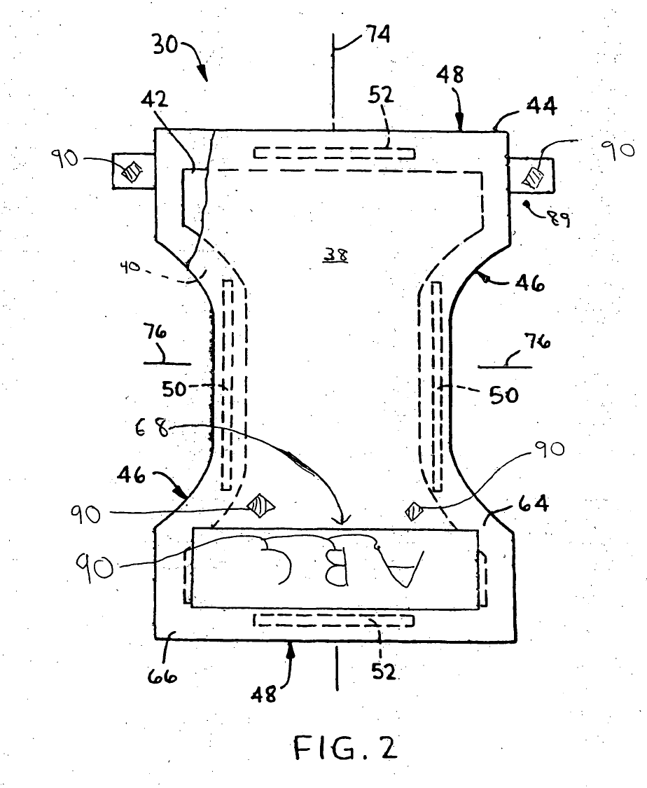

Referring now to the drawings, FIG. 1 illustrates a disposable absorbent article such as a disposable diaper (30) in an unfolded, flat-out, uncontracted state (i.e., with all elastic induced gathering and contraction removed). Portions of the structure are partially cut away to more clearly show the interior construction of the diaper (30), with the surface of the diaper (30) which contacts the wearer facing the viewer. FIGS. 1 and 2 illustrate a disposable diaper (30) as having a front region (32), a rear region (34) and a crotch portion (36) located between the front and rear regions. The diaper (30) comprises a backsheet (38), a topsheet (40), and an absorbent core (42) situated between the backsheet and the topsheet. The outer edges of the diaper (30) define a periphery (44) with transversely opposed, longitudinally extending side edges (46); longitudinally opposed, transversely extending end edges (48); and a system of elastomeric gathering members, such as a system including leg elastics (50) and waist elastics (52). The longitudinal side edges (46) define the leg openings (54) for the diaper (30), and optionally, are curvilinear and contoured. The transverse end edges (48) are illustrated as straight, but optionally, may be curvilinear. The diaper (30) may also comprise additional components to assist in the acquisition, distribution and storage of bodily waste. For example, the diaper (30) may comprise a transport layer, such as described in U.S. Pat. No. 4,798,603, issued to Meyer et al., or a surge management layer, such as described in European Patent Application Publication No. 0 539 703, published May 5, 1993.

The diaper (30) generally defines a longitudinally extending length dimension (56), and a laterally extending width dimension (58), as representatively illustrated in FIG. 1. The diaper (30) may have any desired shape, such as rectangular, I-shaped, a generally hourglass shape, or a T-shape.

The backsheet (38) defines a length and a width which, in the illustrated version, coincide with the length and width of the diaper (30). The absorbent core (42) generally defines a length and width which are less than the length and width of the backsheet (38), respectively. Thus, marginal portions of the diaper (30), such as marginal sections of the backsheet (38), may extend past the transversely opposed, longitudinally extending terminal side edges (60) and/or the longitudinally opposed, transversely extending terminal end edges (62) of the absorbent core (42) to form side margins (64) and end margins (66) of the diaper (30). The topsheet (40) is generally coextensive with the backsheet (38), but may optionally cover an area which is larger or smaller than the area of the backsheet, as desired. The backsheet (38) and topsheet (40) are intended to face the garment and body of the wearer, respectively, while in use. As used herein when describing the topsheet (40) in relation to the backsheet (38) and vice versa, the term “associated” encompasses configurations in which the topsheet is directly joined to the backsheet, and configurations where the topsheet is indirectly joined to the backsheet by affixing portions of the topsheet to intermediate members which in turn are affixed to at least portions of the backsheet. The topsheet (40) and the backsheet (38) can, for example, be joined to each other in at least a portion of the diaper periphery (44) by attachment mechanisms (not shown) such as adhesive bonds, sonic bonds, thermal bonds, pinning, stitching, or a variety of other attachment techniques known in the art, as well as combinations thereof.

The topsheet (40) suitably presents a bodyfacing surface which is compliant, soft feeling, and non-irritating to the wearer's skin. Further, the topsheet (40) may be less hydrophilic than the absorbent core (42), to present a relatively dry surface to the wearer, and is sufficiently porous to be liquid permeable, permitting liquid to readily penetrate through its thickness. A suitable topsheet (40) may be manufactured from a wide selection of web materials, such as porous foams, reticulated foams, apertured plastic films, natural fibers, synthetic fibers (for example, polyester or polypropylene fibers), or a combination of natural and synthetic fibers. The topsheet (40) is suitably employed to help isolate the wearer's skin from liquids held in the absorbent core (42).

Various woven and nonwoven fabrics may be used for the topsheet (40). For example, the topsheet (40) may be composed of a meltblown or spunbonded web of polyolefin fibers. The topsheet (40) may also be a bonded-carded web composed of natural and/or synthetic fibers. The topsheet (40) may be composed of a substantially hydrophobic material, and the hydrophobic material may, optionally, be treated with a surfactant, or otherwise processed, to impart a desired level of wettability and hydrophilicity. Specifically, the topsheet (40) may be a nonwoven, spunbond, polypropylene fabric composed of about 2.8 to about 3.2 denier fibers formed into a web having a basis weight of about 22 gsm and a density of about 0.06 g/cc.

The topsheet (40) may also be surface treated with about 0.3 weight percent of a surfactant mixture that contains a mixture of AHCOVEL Base N-62 surfactant and GLUCOPON 220UP surfactant in about a 3:1 ratio based on a total weight of the surfactant mixture. The AHCOVEL Base N-62 surfactant is purchased from Hodgson Textile Chemicals Inc., a business having offices in Mount Holly, N.C., and comprises a blend of hydrogenated ethoxylated castor oil and sorbitan monooleate in a 55:45 weight ratio. The GLUCOPON 220UP surfactant is purchased from Henkel Corporation, Gulph Mills, Pa., and comprises alkyl polyglycoside. The surfactant may also include additional ingredients such as aloe. The surfactant may be applied by any conventional means, such as spraying, printing, brush coating, foam or the like. The surfactant may be applied to the entire topsheet (40) or may be selectively applied to particular sections of the topsheet, such as the medial section along the longitudinal centerline of a diaper, to provide greater wettability of such sections.

The backsheet (38) may suitably be composed of a material which is either liquid permeable or liquid impermeable. It is generally desirable that the backsheet (38) be formed from a material which is substantially liquid impermeable. For example, a typical backsheet (38) can be manufactured from a thin plastic film or other flexible liquid impermeable material. Moreover, the backsheet (38) may be formed from a polyethylene film having a thickness of from about 0.012 mm (0.5 mil) to about 0.051 mm (2.0 mils). If desirous of presenting the backsheet (38) with a more cloth-like feel, the backsheet may comprise a polyethylene film having laminated to the lower or opposing surface thereof a nonwoven web, such as a spunbond web of polyolefin fibers. For example, a polyethylene film having a thickness of about 0.015 mm (0.6 mil) may have thermally laminated thereto a spunbond web of polyolefin fibers, which fibers have a thickness of about 1.5 to about 2.5 denier per filament, which nonwoven web has a basis weight of about 24 gsm (0.7 osy). Methods of forming such cloth-like outer covers are known to those skilled in the art. Further the backsheet (38) may be a stretchable material, a method of forming such a material may be found in U.S. Pat. No. 5,226,992 issued to Morman, further various examples of extensible materials are described in U.S. Pat. No. 6,264,641 issued to VanGompel et al.; the entire disclosures of which are hereby incorporated by reference in a manner that is consistent herewith

Further, the backsheet (38) may be formed of a woven or nonwoven fibrous web layer which has been totally or partially constructed or treated to impart a desired level of liquid impermeability to selected regions that are adjacent or proximate the absorbent core (42). Still further, the backsheet (38) may optionally be composed of micro-porous “breathable” material which permits vapors to escape from the absorbent core (42) while still preventing liquid exudates from passing through the backsheet.

The absorbent core (42) may comprise a matrix of hydrophilic fibers, such as a web of cellulosic fluff, mixed with particles of a high-absorbency material commonly known as superabsorbent material. In a particular version, the absorbent core (42) comprises a mixture of superabsorbent hydrogel-forming particles and wood pulp fluff. The wood pulp fluff may be exchanged with synthetic polymeric, meltblown fibers or with a combination of meltblown fibers and natural fibers. The superabsorbent particles may be substantially homogeneously mixed with the hydrophilic fibers or may be non-uniformly mixed.

The absorbent core (42) may have any of a number of shapes. For example, the absorbent core (42) may be rectangular, I-shaped or T-shaped. It is often considered as desirable for the absorbent core (42) to be narrower in the crotch portion than the rear or front region(s).

The high-absorbency material can be selected from natural, synthetic and modified natural polymers and materials. The high-absorbency materials can be inorganic materials, such as silica gels, or organic compounds, such as crosslinked polymers. Often, the high-absorbency material is present in the absorbent core (42) in an amount of from about 5 to about 100 weight percent based on total weight of the absorbent core.

The disposable absorbent articles described herein also comprise fasteners (82) for securing the absorbent article about the waist of the wearer. The illustrated versions of the diaper (30) comprise such fasteners (82). In at least one version, the fasteners (82) are situated in the rear region (34) of the diaper (30), and located inboard each longitudinal extending side edge (46). The fasteners (82) may be configured to encircle the hips of the wearer and engage the backsheet (38) of the front region (32) of the diaper (30) for holding the diaper (30) on the wearer. Suitable fasteners are well known to those of skill in the art and can comprise adhesive tape tab fasteners, hook and loop fasteners, mushroom fasteners, snaps, pin, belts and the like, and combinations thereof. Desirably, the fasteners (82) are releasably engageable directly with the garment facing surface of the backsheet (38). Desirably, the fasteners (82) comprise a mechanical fastening system. Alternatively, the diaper (30) may comprise a fastening panel (68) situated in the front region (32) of the garment facing surface of the backsheet (38). In such a configuration, the fasteners (82) are releasably engageable with the fastening panel (68) to maintain the diaper (30) about the waist of the wearer.

A configuration which employs a selectively releasable, inter-engaging mechanical fastening system can, for example, locate the first fastener component on the ear (89), and can locate the cooperating, second fastener component on the fastening panel (68). For example, with the representatively shown hook-and-loop fastener, the fastening component, which is attached to the ear (89), may comprise a hook type of mechanical engagement element, and the complementary fastening component, is the fastening panel (68) which can comprise a loop type of fastening element.

It should also be readily apparent that the relative positions and/or materials of the first fastening component and its cooperating, complementary fastening component can be transposed.

The loop material can comprise a nonwoven, woven or knit fabric. For example, a suitable loop material fabric can be composed of a 2 bar, warp knit fabric of the type available from Guilford Mills, Inc., Greensboro, N.C. under the trade designation #34285, as well as other types of knit fabrics. Suitable loop materials are also available from the 3M Company, which has distributed a nylon woven loop under their SCOTCHMATE brand. The 3M Company has also distributed a linerless loop web with adhesive on the backside of the web, and 3M knitted loop tape.

The disposable diaper (30) of the various aspects of the present invention further includes at least one graphic (90) disposed on the backsheet (38), ear (89) or other portion of the garment facing surface. Suitably, the disposable diaper (30) may include a plurality of graphics (90). The graphics (90) may include, but are not limited to, scenes, characters, animals, objects, alphanumerics such as numbers, letters, words, phrases and the like, highlighting or emphasizing leg openings (54) in order to make product shaping more evident or visible to the user; highlighting or emphasizing areas of the product to simulate functional components such as elastic leg bands, elastic waistbands, simulated “fly openings” for boys, ruffles for girls; highlighting areas of the product to change the appearance of the size of the product; temperature indicators, and the like in the product; registering a back label, or a front label, in the product; and registering written instructions at a desired location in the product.

The graphics (90) can be formed on or applied to the backsheet (38) or another substrate bonded to or placed with or placed near the backsheet (38) by any suitable technique. The graphics (90) are suitably registered with other components of the absorbent article during manufacture such that the graphics (90) are positioned in the desired regions of the product.

Graphics (90) suitable for use with the present invention are described in U.S. Pat. No. 6,297,424 issued Oct. 2, 2001 to Olson, et al. and U.S. Pat. No. 6,307,119 issued Oct. 23, 2001 to Cammarota et al., the disclosures of which are incorporated herein to the extent they are consistent (i.e., not in conflict) herewith.

As mentioned above, the graphics (90) may be disposed on the disposable diaper (30) using a variety of methods. For example, the graphics (90) may suitably be disposed on the disposable diaper (30) by being imprinted thereon using a flexographic printing process. Flexographic printing is a conventional printing technique which uses flexible, raised rubber or photopolymer plates to carry an inked image to a substrate, such as a backsheet (38), a topsheet (40), or an ear (89) of the disposable diaper (30). As an example, flexographic printing apparatus are shown and/or described in U.S. Pat. No. 5,458,590 (Schleinz et al.); U.S. Pat. No. 5,566,616 (Schleinz et al.); U.S. Pat. No. 6,684,772 (Harte); and U.S. Pat. No. 4,896,600 (Rogge et al.). Alternatively, the graphics (90) may suitably be disposed on the disposable diaper (30) by imprinted thereon using a non-contact ink jet printing process. Use of ink jet printing is well suited to producing fine patterns with high detail. Ink jet printing also allows rapid changes to the pattern with only a change in programming versus fabrication of new printing rolls, screens, or plates. As used herein, “non-contact printing” refers to a form of printing in which an image is formed on a substrate without direct contact between the substrate and the apparatus producing the image.

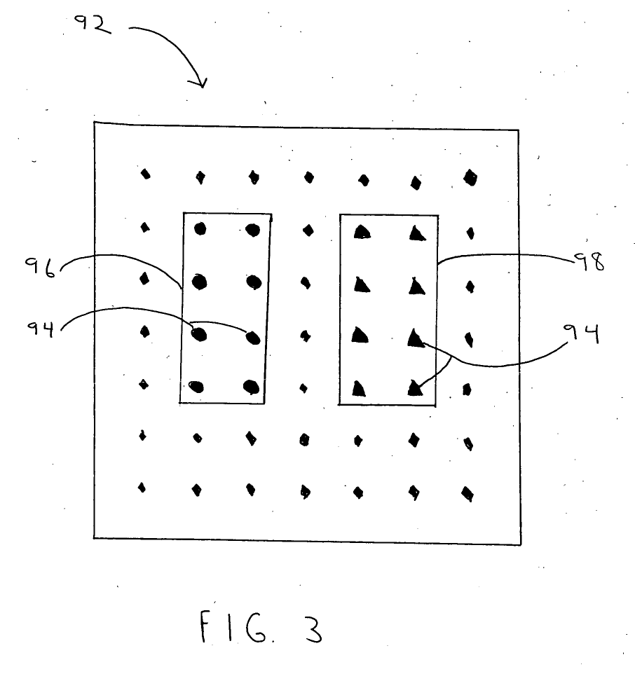

As shown in FIG. 3, the disposable diaper (30) of the various aspects of the present invention further includes a bond pattern (92) disposed on the backsheet (38), ear (89) or other portion of the garment facing surface. As used herein a bond pattern includes a plurality of bond element, and in accordance with the present invention, includes two or more distinct bond regions having differing bond characteristics. The disposable diaper (30) includes a first bond region (96) and a second bond region (98) disposed on the backsheet (38), ear (89) or other portion of the garment facing surface. Each bond region (96, 98) include a bond elements having common bond characteristics. The first bond region (96) and the second bond region (98) each have a distinct bond characteristic, which will be discussed below. The first bond region (96) may be printed, alternatively the second bond region (98) may be printed, further, both bond regions (96, 98) may be printed.

As described above, print quality is affected by the substrate printed. The inventors have determined that by using a bond pattern (92) having a first and second bond region (96, 98), each having a distinct bond characteristic; the graphics (90) may be improved. The appearance of ink on a bond element (94) may appear different than the appearance of ink between bond elements (94). The present inventors have capitalized on this difference to achieve greater visual variety, without additional ink, or ink colors.

The first bond region (96) and the second bond region (98) may have multiple sizes and shapes. The size of the first and second bond regions (96, 98) may be large enough to be easily visible with the naked eye, more specifically; the first and second bond regions (96) may have a size greater than about 0.1 square inches, alternatively, greater than about 0.5 square inches, alternatively, greater than about 1 square inches, and finally, alternatively, greater than about 4 square inches.

FIG. 3 illustrates a bond pattern (92) including a first bond region (96) and a second bond region (98) each having a distinct bond characteristic, bond element shape. The lines indicating the first and second bond pattern (92, 96) (rectangles in FIG. 3 are not themselves considered part of the bond patterns (92, 96). Specifically, the plurality of bond elements (94) which form the first bond region (96) have a bond element shape which is different than the bond element (94) which form the second bond region (96), specifically a circle shape and a triangle shape. Any shape may be utilized, either geometric such as shown, alternatively the shape may relate to the graphic (90) which is printed upon the bond region (96, 98). For example, if the graphic (90) is a dog, the bond elements (94) may be the shape of a dog, or dog bone.

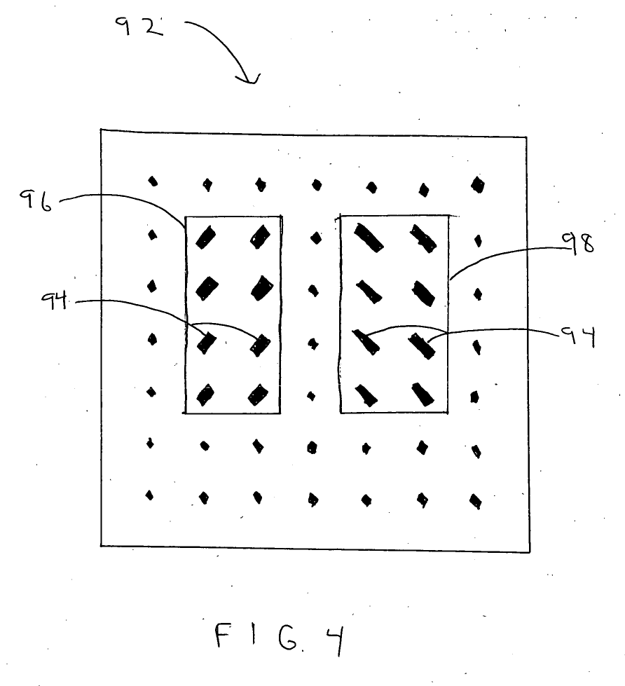

FIG. 4 illustrates a bond pattern (92) including a first bond region (96) and a second bond region (98) each having a distinct bond characteristic. Specifically, the plurality of bond elements (94) which form the first bond region (96) have a bond orientation which is different than the plurality of bond elements (94) which form the second bond region (96). Specifically the bond orientation differs by approximately 90 degrees. The bond orientation of a bond element (94) in the first bond region (96) as compared to the bond orientation of a bond element (94) in the second bond region (98) is defined as the smallest amount of rotation, either clockwise or counterclockwise, that the element (94) in the first bond region (96) must be rotated to align with the bond element (94) in the second bond region (98). These comparisons are described in the field of translational geometry. The bond orientation may differ by greater than about 15 degrees, alternatively, greater than about 45 degrees, alternatively greater than about 90 degrees, or alternatively, greater than about 100 degrees.

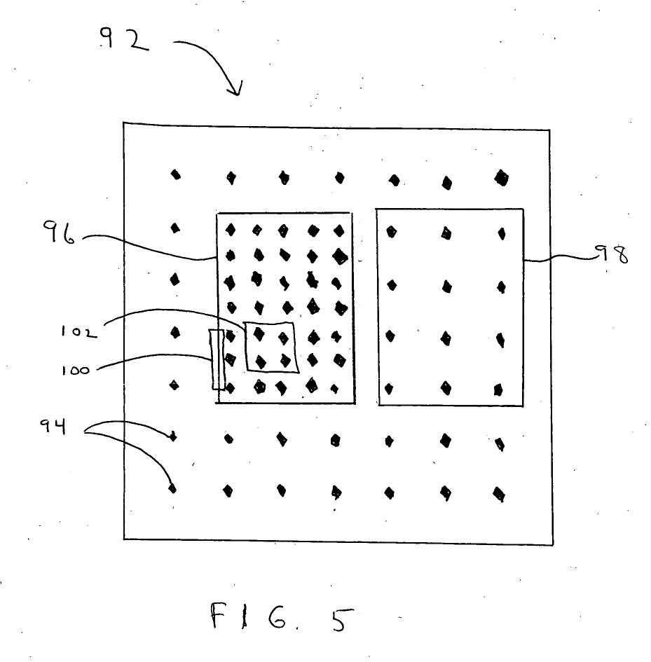

FIG. 5 illustrates a bond pattern (92) including a first bond region (96) and a second bond region (98) each having a distinct bond characteristic. Specifically, the first bond region (96) has a bond area that differs from the bond area of the second bond region (98). The difference in bond area is defined by the absolute value of the difference between the bond area of the first bond region (96) minus the bond area of the second bond region (98). For example if the bond area of the first bond region (96) was 20 percent and the bond area of the second bond region (98) was 5 percent, the bond area of the first bond region (96) would differ from the bond area of the second bond region (98) by 15 percent. The bond area may differ by greater than about 3 percent, alternatively, greater than about 8 percent, alternatively greater than about 12 percent, or alternatively, greater than about 15 percent.

FIG. 5 also illustrates a bond pattern (92) including a first bond region (96) and a second bond region (98) each having a second distinct bond characteristic, specifically bond density. The difference in bond density is defined by the absolute value of the difference between the bond density of the first bond region (96) minus the bond density of the second bond region (98). For example if the bond density of the first bond region (96) was 400 bonds/square inch and the bond density of the second bond region (98) was 100 bonds/square inch, the bond density of the first bond region (96) would differ from the bond density of the second bond region (98) by 300 bonds/square inch. FIG. 5 illustrates how multiple bond characteristics may differ between a first bond region (96) and a second bond region (98). In this way, the print quality of the graphic (90) printed on the bond pattern (92) may be modified to achieve any number of desired aesthetic effects.

FIG. 5 illustrates a first bond region (96) with a portion of the bond region interior (102) and a portion of the bond perimeter (100) highlighted. The bond perimeter (100) is defined as the unbonded border surrounding the bond region. The bond region interior (102) is defined by the area bounded by the bond perimeter (100).

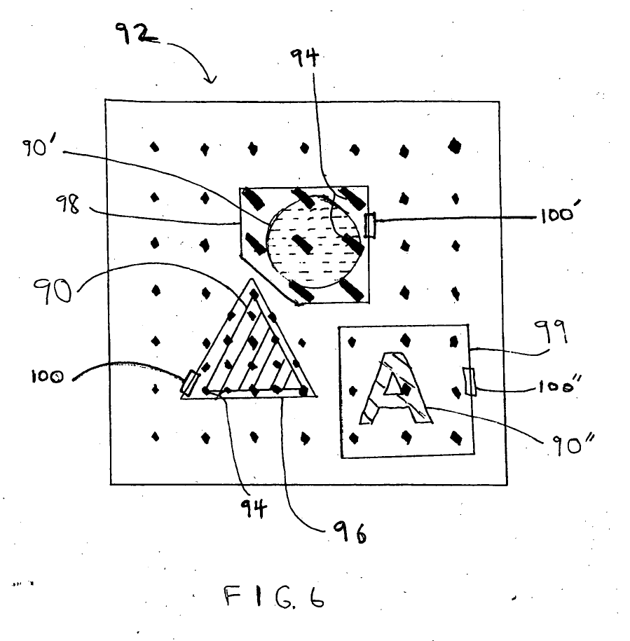

FIG. 6 illustrates a bond pattern (92) with a first bond region (96) and a second bond region (98) and a third bond region (99). The first bond region (96) having a bond perimeter (100) with a graphic (90) (a triangle) located within the bond perimeter of the first bond region (96). The graphic (90) alternatively extend beyond the bond perimeter (96). The second bond region (98) has a bond perimeter (100′) with a graphic (90′) (a circle) located within the bond perimeter of the second bond region (98). Further the third bond region (99) has a bond perimeter (100″) with a graphic (90″) (an “A”) located within the bond perimeter of the third bond region (99).

The first and second bond regions (96, 98, 99) are suitably registered with other components of the absorbent article during manufacture such that the bond regions (96, 98, 99) are positioned in desired regions of the product. Specifically the first bond region (96) may be associated with the leg openings. (56) in order to highlight or emphasize product shaping. The second bond region (98) may be associated with the waist elastics (52) to emphasize the appearance of elastic properties.

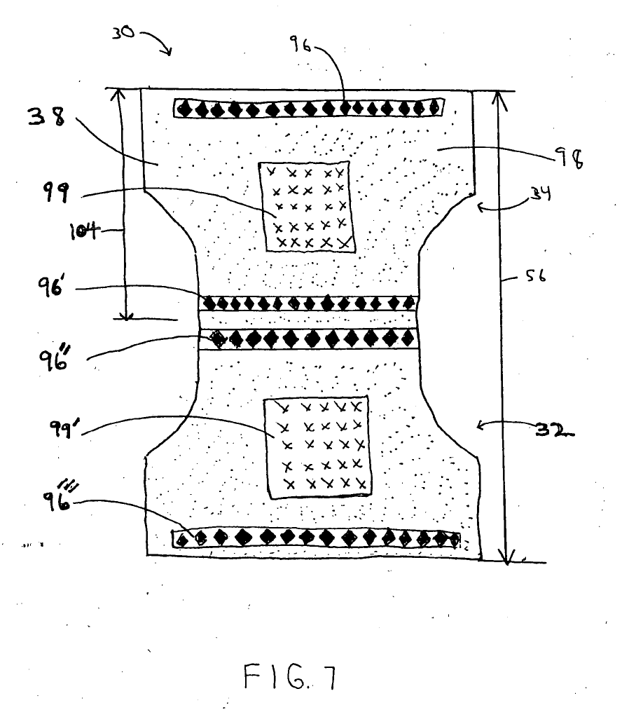

FIG. 7 illustrates a bond pattern (92) disposed on a garment facing surface of a disposable diaper (30) including a first bond region (96), a second bond region (98) and a third bond region (99) each having a distinct bond characteristic. The first bond region (96) includes bond elements in the shape of diamonds arranged in a row. The second bond region (98) includes small point bond elements uniformly distributed on the garment facing surface. The third bond region (99) includes bond elements in the shape of X's arranged in aligned rows and columns.

As illustrated in FIG. 7 the bond pattern (92) includes three areas with similar bond characteristics as the first bond region (96) specifically regions (96′), (96″) and (96′″). The bond pattern (92) includes one area with similar bond characteristics as the third bond region (99), specifically region (99′).

Any combination of the first (96), second (98) and third (99) bond regions may be printed. The first bond region (96) corresponds generally to the rear waist elastic region. The second bond region (98) corresponds generally to the majority of surface area of the garment facing surface. And the third bond region (99) generally corresponds to the central area of the rear region (34), which may include a rear graphic. The bond pattern (92) also has a bond pattern repeat length (104) which is equal to one-half the length dimension of the disposable diaper (30). The bond pattern repeat length (104) may be equal to one, alternatively, one-half, alternatively, one-third, and alternatively one-fourth the length dimension of the disposable diaper (30).

As various changes could be made in the above constructions and methods, without departing from the scope of the invention, it is intended that all matter contained in the above description and shown in the accompanying drawings shall be interpreted as illustrative and not in a limiting sense.

What is claimed is:

1. A disposable absorbent article comprising:
a topsheet;
a backsheet; and
an absorbent core located between the topsheet and the backsheet;
wherein the backsheet comprises a bond pattern which defines a first bond region having a first bond pattern and a first bond perimeter and a second bond region having a second bond pattern and a second bond perimeter, the first bond pattern being unique from the second bond pattern, a first graphic located within the first bond perimeter, and a second graphic located within the second bond perimeter.

2. The disposable absorbent article of claim 1 wherein the first graphic extends beyond the first bond perimeter.

3. The disposable absorbent article of claim 1 wherein the first bond region has a bond area, the second bond region has a bond area and the first bond region bond area differs from the second bond region bond area by greater than about 3 percent.

4. The disposable absorbent article of claim 1 wherein the first bond region has a plurality of bond elements having a first bond orientation, the second bond region has a plurality of bond elements having a second bond orientation, the first bond element orientation differs from the second bond element orientation by greater than about 15 degrees.

5. The disposable absorbent article of claim 1 wherein the first bond region has a bond density, the second bond region has a bond density and the first bond region bond density differs from the second bond region bond density by greater than about 50 bonds/square inch.

6. The disposable absorbent article of claim 1 wherein the first bond region has a plurality of bond elements having a first bond element shape, the second bond region has a plurality of bond elements having a second bond element shape, the first bond element shape differs from the second bond element shape.

7. The disposable absorbent article of claim 1 wherein the backsheet is a nonwoven.

8. A disposable absorbent comprising:
a topsheet;
a backsheet defining a backsheet length; and
an absorbent core located between the topsheet and the backsheet;
wherein the backsheet comprises
a bond pattern which defines a first bond region having a first bond pattern and a first bond perimeter and a second bond region having a second bond pattern and a second bond perimeter, the first bond pattern being unique from the second bond pattern, a first graphic located within the first bond perimeter, and a second graphic located within the second bond perimeter, the bond pattern on the backsheet having a repeat length of one-fourth, one-third, one-half or one times the backsheet length, the first bond pattern is located in a first waist region having a bond pattern area of at least 1 square inch and the second bond pattern is located in a crotch region.

9. The disposable absorbent article of claim 8 wherein the first bond region has a bond area, the second bond region has a bond area and the first bond region bond area differs from the second bond region bond area by greater than about 3 percent.

10. The disposable absorbent article of claim 8 wherein the first bond region has a plurality of bond elements having a first bond orientation, the second bond region has a plurality of bond elements having a second bond orientation, the first bond element orientation differs from the second bond element orientation by greater than about 15 degrees.

11. The disposable absorbent article of claim 8 wherein the first bond region has a bond density, the second bond region has a bond density and the first bond region bond density differs from the second bond region bond density by greater than about 20 bonds/square inch.

12. The disposable absorbent article of claim 8 wherein the first bond region has an area of greater than about 0.01 square inches, and the second bond region has an area of greater than about 0.01 square inches.

13. The disposable absorbent article of claim 8 wherein first bond region has a plurality of bond elements having a first bond element shape, the second bond region has a plurality of bond elements having a second bond element shape, the first bond element shape differs from the second bond element shape.

14. The disposable absorbent article of claim 8 wherein the backsheet is a nonwoven.

15. A disposable absorbent article comprising:
a bodyfacing surface; and
a garment facing surface
wherein the garment facing surface comprises a bond pattern which defines a first bond region having a first unique bond pattern and a first bond perimeter, a second bond region having a second unique bond pattern and a second bond perimeter, and a third bond region having a third unique bond pattern and a third bond perimeter, a first graphic located within the first bond perimeter, a second graphic located within the second bond perimeter and a third graphic located within the third bond perimeter.

16. The disposable absorbent article of claim 15 wherein the first bond region has a bond area, the second bond region has a bond area and the first bond region bond area differs from the second bond region bond area by greater than about 3 percent.

17. The disposable absorbent article of claim 15 wherein the first bond region has plurality of bond elements having a first bond orientation, the third bond region has a plurality of bond elements having a second bond orientation, the first bond element orientation differs from the third bond element orientation by greater than about 15 degrees.

18. The disposable absorbent article of claim 15 wherein the second bond region has a bond density, the third bond region has a bond density and the second bond region bond density differs from the third bond region bond density by greater than about 20 bonds/square inch.

19. The disposable absorbent article of claim 15 wherein the first bond region has a plurality of bond elements having a first bond element shape, the second bond region has a plurality of bond elements having a second bond element shape, the first bond element shape differs from the second bond element shape.

20. The disposable absorbent article of claim 15 wherein the garment facing surface is a nonwoven.

21. A disposable article comprising a nonwoven, wherein the nonwoven comprises a bond pattern which defines a first bond region having a first bond pattern and a first bond perimeter and a second bond region having a second bond pattern and a second bond perimeter, the first bond pattern being unique from the second bond pattern, a first graphic located within the first bond perimeter, and a second graphic located within the second bond perimeter.
